(19) United States
(12) Patent Application Publication
(10) Pub. No.: US 2026/0207141 A1
(43) Pub. Date: Jul. 23, 2026

(54) IMPLANTABLE SENSOR ASSEMBLY INCLUDING A SENSOR AND A STIFFENED STRUCTURE

(71) Applicant: MY01 IP HOLDINGS INC., Montreal (CA)

(72) Inventors: Mohamad Nizar KEZZO, Montreal (CA); Maneesha Pramodh Karunagoda KALAHE PADIKORALLAGE, Verdun (CA); Anya JESSON, Montreal (CA); Samuel Anjolaoluwa DAYO, Montreal (CA); Rachel TCHINOV, Montreal (CA); Oliver MILLER, Montreal (CA); Animesh SAHA SHOVAN, Montreal (CA); Cédric VALLE-MENA, Montreal (CA); Claudio BUONAMICI, Montreal (CA)

(21) Appl. No.: 19/140,434

(22) PCT Filed: Dec. 19, 2023
(86) PCT No.: PCT/CA2023/051699

§ 371 Date: Jun. 18, 2025

Related U.S. Application Data
(60) Provisional application No. 63/476,266, filed on Dec. 20, 2022.

(51) Int. Cl. A61B 5/00

(52) U.S. Cl. CPC A61B 5/686; A61B 2560/063

(57) ABSTRACT

An implantable sensor assembly for taking measurements within the body is disclosed, wherein the implantable sensor assembly includes a board, a first packaged sensor which is mounted to the board, a stiffened structure and a fastening material. The first packaged sensor includes a first sensing element, an application-specific integrated circuit (ASIC), a supportive medium and an exterior package. The fastening material joins at least the stiffened structure to the board and the fastening material exposes, at least in part, the first packaged sensor to the external environment.

The present application claims priority from U.S. provisional patent application No. 63/476,266 filed on Dec. 20, 2022, incorporated herein by reference.

### TECHNICAL FIELD

The present disclosure relates to the field of implantable sensors. More specifically, the present disclosure relates to implantable biocompatible sensor assemblies.

### BACKGROUND

Implantable biocompatible sensor assemblies are adapted to be inserted under a subject's skin in order to measure and collect data related to physical conditions of the subject body. An implantable sensor assembly may include one or more sensors capable of measuring conditions that prevail in the body, for example temperature, pressure, partial pressure of oxygen, potential hydrogen (pH), and the like. The sensor assembly may be adapted to measure one or more of these conditions, but its measurements may be altered by the other of these conditions. Thus, reducing the impact of unrelated physiological conditions on sensor readings is a major driver of sensor development.

Implantable biocompatible sensor assemblies have undergone extensive improvements over the years and offer a rapid and accurate way for doctors, nurses and caregivers to monitor subjects with particular medical conditions. An example of a method and system for installing a sensor assembly in a body is provided in WO 2018/167693 A1 by Harvey et al., the disclosure of which is incorporated by reference herein. Once a sensor assembly is in place under the skin of patient, it may provide measurements to an external module via signals that propagate, for example, through a communication cable.

### SUMMARY

The present disclosure relates to an implantable sensor assembly that is configured to take measurements within the body, including but not limited to pressure, partial pressure of oxygen, and temperature measurements.

An implantable sensor assembly with such an aim is described in PCT patent application No. PCT/CA2021/050041, comprising a support structure including a board, a compliant structure disposed on a top surface of the board, and a sensor supported by the compliant structure above the top surface of the board. An aperture is formed in the support structure for exposing at least in part a face of the sensor. The sensor may be a pressure sensor having a sensing membrane exposed through the aperture formed in the support structure. A stiffener, which may be conductive, may be mounted to a bottom surface of the board. The sensor and other components may be covered by a polymer shell having a conductive cover or by a gel contained within a rigid cap, which may be conductive. An electromagnetic shield may be formed by an electrical connection between the conductive cover or the conductive rigid cap and the conductive stiffener. The compliant structure aims to isolate the sensing element from mechanically-induced stress, which may be introduced by temperature coefficient of expansion mismatches, or by externally applied forces. Unfortunately, the sensing element must be directly attached to the board and thus the compliant structure cannot completely isolate the sensing element from external stresses, resulting in a noisy signal. Additionally, the free-form compliant structure necessitates complex manufacturing, as it may be composed of multiple gels/epoxies which may be difficult to apply consistently. For instance, when fabricating the implantable sensor assembly of PCT patent application No. PCT/CA2021/050041, the compliant structure material is poured over the sensing element with sufficient flowability to fully cover the sensing element. Without a bounding volume to consistently limit the flow of the compliant structure material, manufacturing yields can be low, and overflow onto other sensitive components in the assembly may be common.

In the present disclosure, an implantable sensor assembly without a compliant structure, a polymer shell or a cap is described, thereby eliminating complex manufacturing steps and improving sensor performance. In the present disclosure, a first packaged sensor including an exterior package which encloses a sensing element is described. The first packaged sensor may be mounted on a board (i.e. a printed circuit board) using standard surface mounting technology. Additionally, the board is joinable to a stiffened structure.

In accordance with an embodiment of the invention there is provided an implantable sensor assembly for taking measurements within the body, including: a board; a first packaged sensor, mounted to a top surface of the board, the first packaged sensor including: a first sensing element; an application-specific integrated circuit (ASIC), wherein the first sensing element is operatively connected to the ASIC; a supportive medium; and an exterior package containing the first sensing element, the ASIC and the supportive medium; a stiffened structure, wherein the stiffened structure further includes at least one set of through apertures between a top surface of the stiffened structure and a bottom surface of the stiffened structure, wherein a first set of through apertures of the stiffened structure surrounds the perimeter of the first packaged sensor; and a fastening material, wherein the fastening material joins at least the stiffened structure to the top surface of the board and wherein the fastening material exposes, at least in part, the first packaged sensor to an external environment.

In some embodiments, the board may include a printed circuit board (PCB).

In some embodiments, the board may include one or more metal pads on the top surface of the board, and wherein the one or more metal pads may be configured to increase the adhesion of the fastening material to the top surface of the board.

In some embodiments, a top face of the first packaged sensor may be at or below the top surface of the stiffened structure.

In some embodiments, the first packaged sensor may include a surface mounted technology (SMT) sensor.

In some embodiments, the first packaged sensor may include a pressure sensor, and wherein the first sensing element of the first packaged sensor may be exposed to the external environment by at least one hole in the top face of the first packaged sensor.

In some embodiments, the fastening material may be applied, at least in part, to (i) the interface between the stiffened structure and the board, and (ii) the interface between the stiffened structure and the exterior package of the first packaged sensor, other than the top face of the first packaged sensor, such that the top face of the first packaged sensor may be at least mostly free of the fastening material.

In some embodiments, the fastening material may include a biocompatible material.

In some embodiments, the fastening material may include viscosity with temperature.

In some embodiments, the fastening material may include at least one hard epoxy.

In some further embodiments, the at least one hard epoxy further may include a first hard epoxy with a first viscosity, and a second hard epoxy with a second viscosity greater than the first viscosity.

In some embodiments, the stiffened structure may be configured to enable the fastening material to be applied via one or a combination of (i) one or more injection holes and (ii) at least one set of through apertures, and wherein the flow of the fastening material may be constrained, at least in part, by the stiffened structure and the board.

In some embodiments, a forward end of the stiffened structure may include a hook hole, and wherein the hook hole may be adapted for interfacing with an insertion device.

In some embodiments, the cross-sectional area of the forward end of the stiffened structure may be less than the maximum cross-sectional area of the stiffened structure.

In some embodiments, the stiffened structure may be made from a biocompatible material.

In some embodiments, the sensor assembly may be conformally coated in a layer of a biocompatible material, wherein the layer of biocompatible material conformally may coat, at least partly, the interior of the first packaged sensor, and wherein the layer of biocompatible material may be one from a selection of (i) parylene, (ii) a biocompatible epoxy and (iii) a biocompatible gel.

In some embodiments, the first packaged sensor may be conformally coated in a layer of biocompatible material, wherein the layer of biocompatible material conformally may coat, at least partly, the interior of the first packaged sensor, and wherein the layer of biocompatible material may be one from a selection of (i) parylene, (ii) a biocompatible epoxy and (iii) a biocompatible gel.

In some embodiments, the implantable sensor assembly may include: at least one peripheral SMT component, mounted to the board, wherein the at least one peripheral SMT component may be operatively connected to the first packaged sensor; and a communication cable extending from the board and adapted to operatively connect the first packaged sensor to a data collection module.

In some embodiments, the board and the communication cable may include the same PCB, wherein the PCB may be flexible.

In some further embodiments, electrical connections between the at least one peripheral SMT component and the first packaged sensor each may include one or more elements selected from wire bonds, solder paste traces on the board, conductive ink traces on the board, conductive metal traces on the board and conductive epoxy traces on the board.

Additionally, in the present disclosure, there is provided an implantable multi-sensor assembly for taking measurements within the body, including: a board; a first packaged sensor, mounted to a top surface of the board, the first packaged sensor including: a first sensing element; an application-specific integrated circuit (ASIC), wherein the first sensing element is operatively connected to the ASIC; a supportive medium; and an exterior package containing the first sensing element, the ASIC and the supportive medium; a second sensor, mountable to the top surface of the board; a stiffened structure, wherein the stiffened structure further includes at least one set of through apertures between a top surface of the stiffened structure and a bottom surface of the stiffened structure, wherein a first set of through apertures surrounds the perimeter of the first packaged sensor and wherein a second set of through apertures surrounds the perimeter of the second sensor; and a fastening material, wherein the fastening material joins at least the stiffened structure to the top surface of the board and wherein the fastening material exposes, at least in part, the first packaged sensor and the second packaged sensor to the external environment.

In some further embodiments, the second sensor may include one from a set of (i) an optoelectronic sensor, (ii) a glucose sensor, (iii) a biomarker sensor, (iv) a pH sensor, (v) a temperature sensor.

In some further embodiments, the optoelectronic sensor may include: at least one photodiode; a first micro-light emitting diode (μLED); a second μLED.

In some further embodiments, the at least one photodiode, the first μLED and the second μLED may include at least one packaged surface mounted technology (SMT) component mounted to the board.

In some further embodiments, the at least one photodiode may be configured to measure the light emitted at a first wavelength by the first μLED and at a second wavelength by the second μLED and attenuated by a tissue in a body.

In some further embodiments, the first wavelength and the second wavelength may include one from (i) infrared and (ii) red spectrum.

In some further embodiments, the fastening material may be configured to scatter light emitted by the first μLED and the second μLED through a tissue.

In some embodiments, the board further may include a PCB.

In some embodiments, the board further may include one or more metal pads on the top surface of the board, and wherein the one or more metal pads may be configured to increase the adhesion of the fastening material to the top surface of the board.

In some embodiments, a top face of the first packaged sensor may be at or below the top surface of the stiffened structure.

In some embodiments, the first packaged sensor may include a surface mounted technology (SMT) sensor and wherein the second sensor further may include a SMT sensor.

In some embodiments, the first packaged sensor may include a pressure sensor, and wherein the first sensing element of the first packaged sensor may be exposed to the external environment by at least one hole in the top face of the first packaged sensor.

In some embodiments, the fastening material may include a biocompatible material.

In some embodiments, the fastening material may change viscosity with temperature.

In some embodiments, the fastening material may be optically clear and wherein the fastening material may be applied, at least in part, to (i) the interface between the stiffened structure and the board, and (ii) the interface between the stiffened structure and the exterior package of the first packaged sensor, other than the top face of the first packaged sensor, such that the top face of the first packaged sensor may be at least mostly free of the fastening material and (iii) the interface between the stiffened structure and the second sensor, such that the second sensor may be completely covered by the fastening material.

In some embodiments, the fastening material may include a first hard epoxy and a second hard epoxy, wherein the second hard epoxy may be an optically clear epoxy, and wherein the first hard epoxy may be applied, at least in part, to (i) the interface between the stiffened structure and the board, and (ii) the interface between the stiffened structure and the exterior package of the first packaged sensor, other than the top face of the first packaged sensor, such that the top face of the first packaged sensor may be at least mostly free of the first hard epoxy, and wherein the second hard epoxy may be applied to the interface between the stiffened structure and the second sensor, such that the second sensor may be completely covered by the second hard epoxy.

In some further embodiments, the first hard epoxy may have a first viscosity, wherein the second hard epoxy may have a second viscosity, and wherein either (i) the first viscosity may be greater than the second viscosity or (ii) the second viscosity may be greater than the first viscosity.

In some embodiments, the stiffened structure may be configured to enable the fastening material to be applied via one or a combination of (i) one or more injection holes and (ii) at least one through aperture, and wherein the flow of the fastening material may be constrained, at least in part, by the stiffened structure and the board.

In some embodiments, a forward end of the stiffened structure may include a hook hole and wherein the hook hole is adapted for interfacing with an insertion device.

In some embodiments, the cross-sectional area of the forward end of the stiffened structure may be less than the maximum cross-sectional area of the stiffened structure.

In some embodiments, the stiffened structure may be made from a biocompatible material.

In some embodiments, the sensor assembly may be conformally coated in a layer of biocompatible material, wherein the layer of biocompatible material conformally may coat, at least partly, the interior of the first packaged sensor, and wherein the layer of biocompatible material may be one from a selection of (i) parylene, (ii) a biocompatible epoxy and (iii) a biocompatible gel.

In some embodiments, one or both of the first packaged sensor and the second sensor may be conformally coated in a layer of biocompatible material, wherein the layer of biocompatible material conformally may coat, at least partly, one or both of the interior of the first packaged sensor, and wherein the layer of biocompatible material may be one from a selection of (i) parylene, (ii) a biocompatible epoxy and (iii) a biocompatible gel.

In some embodiments, the implantable sensor assembly may further include: at least one peripheral SMT component, mounted to the board, wherein the at least one peripheral SMT component may be operatively connected to the first packaged sensor; and a communication cable extending from the board and adapted to operatively connect at least the first packaged sensor and the second sensor to a data collection module.

In some further embodiments, the board and the communication cable may include the same PCB, wherein the PCB is flexible.

In some further embodiments, electrical connections between one or a combination of (i) the at least one peripheral SMT component, (ii) the second sensor and (iii) the first packaged sensor may include one or more elements selected from wire bonds, solder paste traces on the board, conductive ink traces on the board, conductive metal traces on the board, conductive epoxy traces on the board, and a combination thereof.

In some further embodiments, the second sensor may include a plurality of components mounted to the board, and wherein each respective through aperture from the second set of through apertures may surround the perimeter of a respective component from the plurality of components mounted to the board.

Additionally, in the present disclosure, there is provided a method of implanting a sensor assembly, including at least one packaged sensor, into a body of a patient including: introducing a sensor assembly into the patient, wherein a stiffened structure is provided to (i) reduce strain on a board of the sensor assembly during the insertion to avoid damage to the board and (ii) at least partly enclose the at least one packaged sensor of the sensor assembly to avoid damage to the at least one packaged sensor.

Additionally, in the present disclosure, there is provided a kit for manufacturing a sensor assembly including: at least one packaged sensors; a stiffened structure that has been adapted to provide, for each of the at least one packaged sensors, a through aperture to accommodate dimensions of the respective packaged sensor; and a board that is adapted to receive the at least one packaged sensors and the stiffened structure.

Additionally, in the present disclosure, there is provided a method of manufacturing the sensor assembly, including: providing a board with at least one packaged sensors mounted thereon; aligning a stiffened structure with the at least one packaged sensors such that the at least one packaged sensors fit into one or more through apertures of the stiffened structure; and fastening the stiffened structure to the board with at least one packaged sensors mounted thereon.

Another broad aspect is an implantable sensor assembly for taking measurements within the body. The sensor assembly includes a board; a first packaged sensor, mounted to a top surface of the board, the first packaged sensor comprising: a first sensing element; an integrated circuit component, wherein the first sensing element is operatively connected to the integrated circuit component; a supportive medium; and an exterior package containing the first sensing element, the integrated circuit component and the supportive medium; a stiffened structure, wherein the stiffened structure further comprises at least one set of through apertures between a top surface of the stiffened structure and a bottom surface of the stiffened structure, wherein a first set of through apertures of the stiffened structure surrounds the perimeter of the first packaged sensor; and a fastening material, wherein the fastening material joins at least the stiffened structure to the top surface of the board and wherein the fastening material exposes, at least in part, the first packaged sensor to an external environment.

In some embodiments, the integrated circuit component may be an application-specific integrated circuit (ASIC).

### BRIEF DESCRIPTION OF THE DRAWINGS

Embodiments of the disclosure will be described by way of example only with reference to the accompanying drawings, in which:

FIG. 1A is an exploded perspective view of an implantable sensor assembly;

FIG. 1B is a perspective view of an implantable sensor assembly;

FIG. 1C is a top plan view of an implantable sensor assembly;

FIG. 1D is a cross-sectional, side elevation view of an implantable sensor assembly;

FIG. 1E is a view of the interior of a first packaged sensor;

FIG. 2A is a perspective view showing the manufacturing process of an implantable sensor assembly;

FIG. 2B is an example of the implantable sensor assembly 100 with improved adhesion and reduced force of insertion;

FIG. 3A is an exploded perspective view of an implantable multi-sensor assembly;

FIG. 3B is a top plan view of an implantable multi-sensor assembly;

FIG. 3C is an exploded perspective view of an implantable multi-sensor assembly, where a second sensor is an optoelectronic sensor;

FIG. 3D is a perspective view of an implantable multi-sensor assembly, where a second sensor is an optoelectronic sensor;

FIG. 3E is a top plan view of an implantable multi-sensor assembly, where a second sensor is an optoelectronic sensor;

FIG. 3F is a cross-sectional, side elevation view of an implantable multi-sensor assembly, where a second sensor is an optoelectronic sensor;

FIG. 4 is a perspective view showing the manufacturing process of an implantable multi-sensor assembly;

FIG. 5A provides an example of a circuit for signal generation and processing, for use with an optoelectronic sensor;

FIG. 5B provides an example of a circuit for signal processing, for use with a signal generated by at least one photodiode;

FIG. 5C provides an example of a circuit for signal generation and processing applied in combination with a packaged sensor;

FIG. 5D provides an example of a circuit for signal generation and processing in combination with a pre-packaged analog front-end (AFE).

FIG. 6A is a perspective view of the implantable sensor assembly and of a forward end of an insertion device, unassembled according to an embodiment;

FIG. 6B is a perspective view of the implantable sensor assembly and of the forward end of the insertion device, assembled according to an embodiment;

### DETAILED DESCRIPTION

Various aspects of the present disclosure generally address one or more of the problems caused by the exposure of an implantable biocompatible sensor assembly to various physical conditions. Additional details of the construction of the implantable sensor assembly and of its various embodiments will be described in relation to the following drawings. In the drawings, the term “forward end” refers to an end of the implantable sensor assembly that is first inserted when in use and the term “rear end” refers to an opposite end of the implantable sensor assembly. The terms “upper”, “lower”, “top” and “bottom” are relative terms defined in relation to the drawings. The skilled reader will appreciate that, in use, the implantable sensor assembly may be implanted sideways or upside down in a body. Furthermore, the skilled reader will appreciate that the implantable sensor assembly may be installed in the body in a multitude of ways, including but not limited to the device disclosed by Harvey in WO 2018/167693 A1. Unless otherwise noted, the drawings are not to scale.

When implanted in a body, a sensor is subject to various conditions that might impact its measurement capabilities. In one example, medical device developers have noted that the structure surrounding the sensor and the sensor itself may expand at different rates as the temperature is modified by insertion in the body due to the difference in the thermal expansion coefficient of the constituent materials. This may induce stress on the sensor and impact its capability to reliably measure parameters of the body. For instance, a pressure sensor inserted into an area of the body might lose sensitivity due to the stresses caused by different expansion rates of the sensor and its supporting structure, which might not be distinguishable from the actual pressure within the body. In another example, a change of dielectric constant due to the insertion of a sensor into the body may adversely affect a measurement signal generated by the sensor. Thus, packaging the sensor may serve to reduce the sensitivity of the sensor to the surrounding environment. Off-the-shelf surface-mount technology (SMT) packaged sensors are a particularly useful class of packaged sensor, as they may be mounted directly onto the surface of a flexible or rigid printed circuit board.

### I. Implantable Sensor Assembly

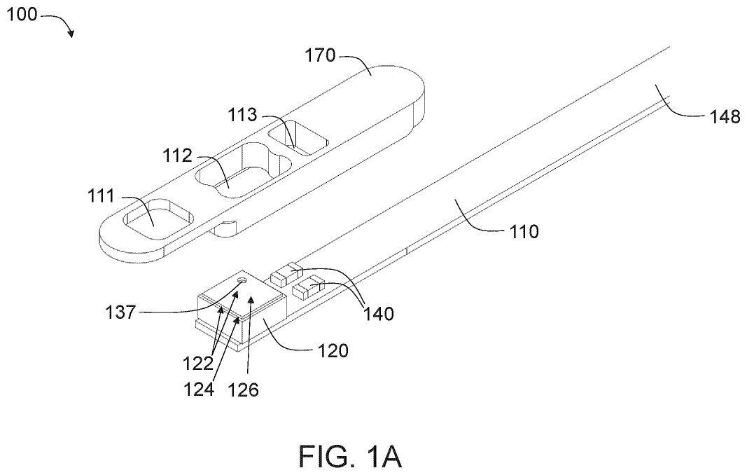

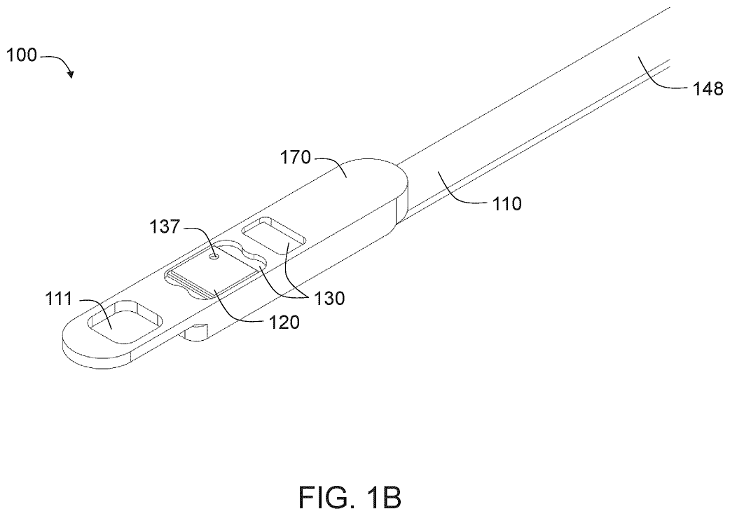

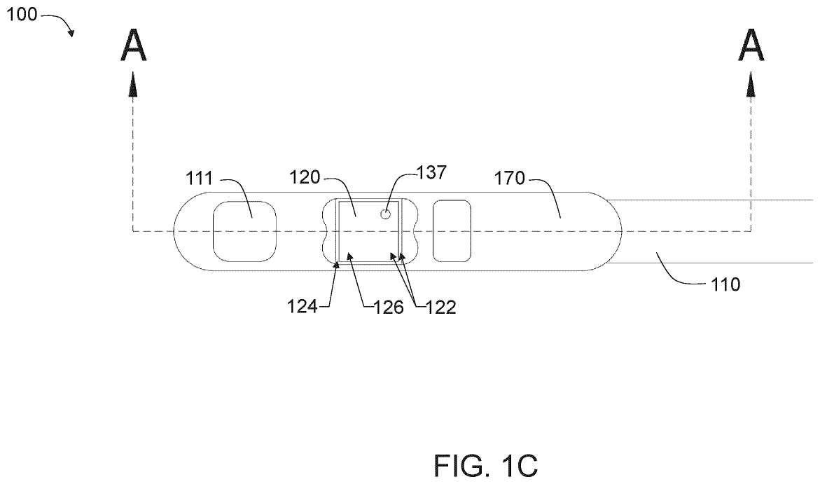

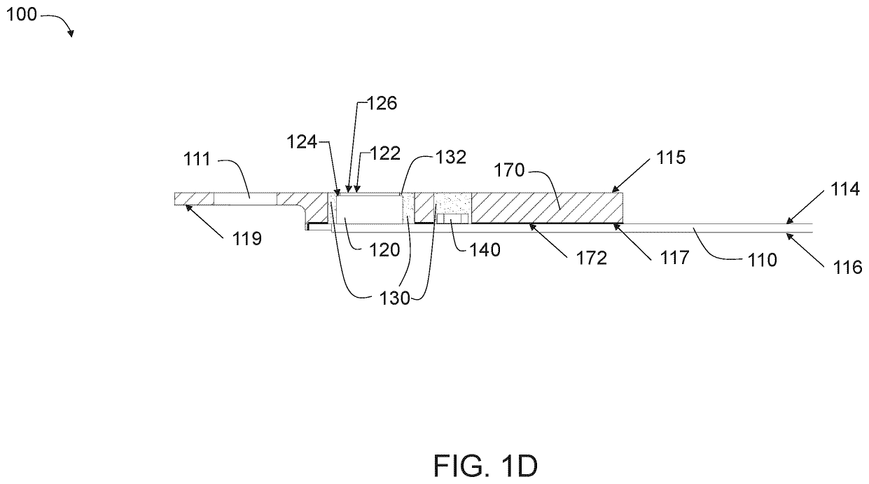

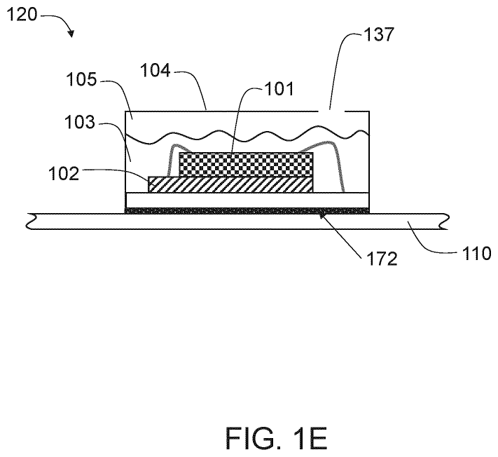

FIG. 1A is an exploded perspective view of an implantable sensor assembly 100. FIG. 1B is a perspective view of the implantable sensor assembly 100 of FIG. 1A. FIG. 1C is a top plan view of an implantable sensor assembly 100. FIG. 1D is a cross-sectional, side elevation view of the implantable sensor assembly 100 along the cross-section A-A shown in FIG. 1C. FIG. 1E is a view of the interior of a first packaged sensor 120.

Referring to FIGS. 1A-E, the implantable sensor assembly 100 includes a board 110, a first packaged sensor 120, a stiffened structure 170 and a fastening material 130. The first packaged sensor 120 is mounted to a top surface 114 of the board 110. The first packaged sensor 120 includes a first sensing element 101, an integrated circuit component 102, (for purposes of illustration, the integrated circuit component 102 will be an application-specific integrated circuit (ASIC) 102, however, it will be understood that other integrated circuit components, such as a micro controller unit (MCU), or an integrated field-programmable gate array (FPGA), may be contemplated), a supportive medium 103 and an exterior package 104. The first sensing element 101 is operatively connected to the ASIC 102. The stiffened structure 170 has a top surface 115 and a bottom surface 117. The stiffened structure 170 has at least one set of through apertures extending between the top surface 115 and the bottom surface 117. A first set of through apertures 112 surrounds the perimeter of the first packaged sensor 120. The fastening material 130 joins at least the stiffened structure 170 to the top surface 114 of the board 110. The fastening material 130 exposes, at least in part, the first packaged sensor 120 to an external environment.

The board 110 provides a surface for mounting at least the first packaged sensor 120. The board 110 is operatively connected to a communication cable 148. The board 110 has the top surface 114 and a bottom surface 116.

The first packaged sensor 120 isolates the first sensing element 101 of the first packaged sensor 120 from stress propagating through the implantable sensor assembly 100. The exterior package 104 of the first packaged sensor 120 is rigid to prevent excessive stress on the supportive medium 103. The supportive medium 103 is a compliant material, and isolates the first sensing element 101 and the ASIC 102 from propagating stress. Together the supportive medium 103, the first sensing element and the ASIC 102 may not fill the entire volume inside the exterior package, leaving an empty volume 105 in the interior of the first packaged sensor 120. The first packaged sensor 120 has a top face 122 including a central area 126 surrounded by a perimeter 124. Additionally, the first packaged sensor 120 may have at least one hole 137 in the top face 122, to expose the first sensing element 101 to the external environment for measurement. To maintain the performance of the first packaged sensor 120, the central area 126 should remain at least mostly free of the fastening material 130. However, lips 132 of the fastening material 130 may extend between the perimeter 124 and the top surface 115.

The stiffened structure 170 is rigid to prevent excessive bending or torsional strain inducing stress in the first packaged sensor 120.

The fastening material 130 is configured to rigidly adhere at least the stiffened structure 170 to the board 110 without failure due to delamination, peeling, shear or tension. Furthermore, the fastening material serves to electrically insulate the electrical components from the external environment. In some embodiments, the board 110 is a printed circuit board (PCB). The PCB may be rigid or flexible.

In some embodiments, the board 110 includes one or more metal pads 244 on the top surface 114 of the board 110, and the one or more metal pads 244 are configured to increase the adhesion of the fastening material 130 to the top surface 114 of the board 110.

In some embodiments, the top face 122 of the first packaged sensor 120 is at or below the top surface 115 of the stiffened structure 170.

In some embodiments, the first packaged sensor 120 is a surface mounted technology (SMT) sensor.

In some embodiments, the first packaged sensor 120 is a pressure sensor, and wherein the first sensing element 101 of the first packaged sensor 120 is exposed to the external environment by at least one hole 137 in the top face 122 of the first packaged sensor 120.

In some embodiments, the fastening material 130 is applied, at least in part, to (i) the interface between the stiffened structure 170 and the board 110, and (ii) the interface between the stiffened structure 170 and the exterior package 104 of the first packaged sensor 120, other than the top face 122 of the first packaged sensor 120, such that the top face 122 of the first packaged sensor 120 is at least mostly free of the fastening material 130. In some further embodiments, the fastening material 130 is at least one hard epoxy. The fastening material 130 may be formed from one or more materials capable of strong bonding to metallic surfaces, such as LOCTITE-3984 from Henkel, MED-302-3M from Epo-Tek, EP30-4Med from Masterbond, or EP42HT-4AOMed from Masterbond. Additionally, due to the envisioned medical application of the implantable sensor assembly 100, the fastening material 130 may be biocompatible.

In some embodiments, the fastening material 130 is a biocompatible material.

In some embodiments, the fastening material 130 changes viscosity with temperature.

In some embodiments, the fastening material 130 is at least one hard epoxy.

In some further embodiments, the at least one hard epoxy further includes a first hard epoxy with a first viscosity, and a second hard epoxy with a second viscosity greater than the first viscosity.

In some embodiments, the stiffened structure 170 is configured to enable the fastening material 130 to be applied via one or a combination of (i) one or more injection holes (not shown in FIG. 1A-E) and (ii) at least one set of through apertures, and wherein the flow of the fastening material is constrained, at least in part, by the stiffened structure and the board.

In some embodiments, a forward end 119 of the stiffened structure 170 further includes a hook hole 111, and wherein the hook hole 111 is adapted for interfacing with an insertion device.

In some embodiments, the cross-sectional area of the forward end 119 of the stiffened structure 170 is less than the maximum cross-sectional area of the stiffened structure. The reduction in cross-sectional area may reduce the force required to pierce the skin and implant the implantable sensor assembly 100.

In some embodiments, the stiffened structure 170 is made from a biocompatible material. For example, the stiffened structure 170 may be stainless steel or titanium.

In some embodiments, the implantable sensor assembly 100 is conformally coated in a layer of biocompatible material, wherein the layer of biocompatible material conformally coats, at least partly, the interior of the first packaged sensor, and wherein the layer of biocompatible material is one from a selection of (i) parylene, (ii) a biocompatible epoxy and (iii) a biocompatible gel.

In some embodiments, the first packaged sensor 120 is conformally coated in a layer of biocompatible material, wherein the layer of biocompatible material conformally coats, at least partly, the interior of the first packaged sensor, and wherein the layer of biocompatible material is one from a selection of (i) parylene, (ii) a biocompatible epoxy and (iii) a biocompatible gel.

In some embodiments, the implantable sensor assembly 100 further includes: at least one peripheral SMT component 140, mounted to the board 110, wherein the at least one peripheral SMT component 140 is operatively connected to the first packaged sensor 120; and the communication cable 148 extending from the board 110 and adapted to operatively connect the first packaged sensor 120 to a data collection module (not shown).

In some further embodiments, the board 110 and the communication cable 148 include the same PCB, wherein the PCB is flexible. The PCB may be rigid or flexible. The PCB made be at least mostly made of polyimide.

In some further embodiments, the electrical connections between the at least one peripheral SMT component 140 and the first packaged sensor 120 each include one or more elements selected from wire bonds, solder paste traces on the board, conductive ink traces on the board, conductive metal traces and conductive epoxy traces on the board 110.

In some embodiments, the board 110 further includes a set of alignment cutouts 242 and the stiffened structure 170 further includes a set of alignment features 243, and wherein the set of alignment cutouts 242 and the set of alignment features 243 are configured to align.

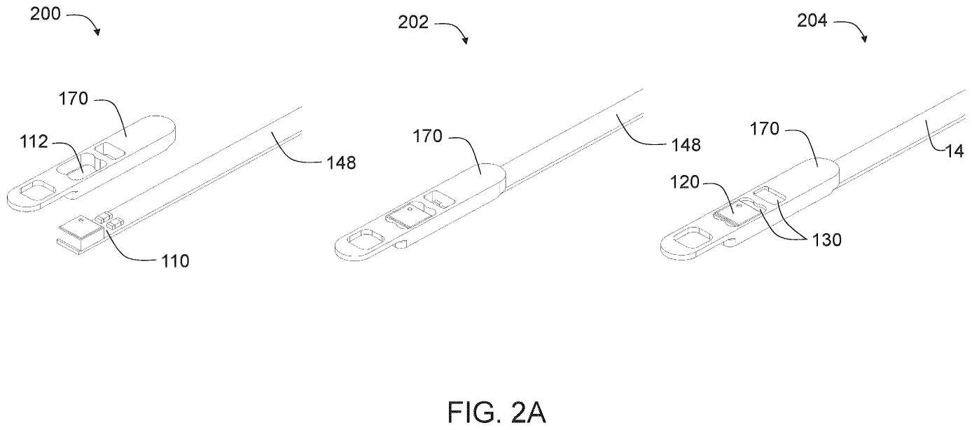

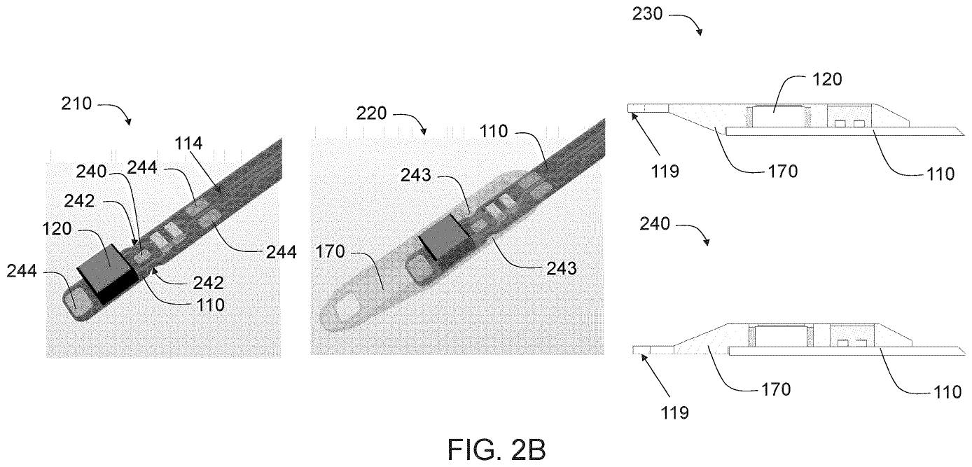

FIG. 2A provides an overview of the fabrication steps of the implantable sensor assembly 100. Schematic 200 is a perspective view of an implantable sensor assembly 100 before assembly. Schematic 202 is a perspective view of an implantable sensor assembly 100 before fastening material 130 is applied. Schematic 204 is a perspective view of an implantable sensor assembly 100 after fastening material 130 is applied.

As shown in schematic 200, when fabricating the implantable sensor assembly 100, the stiffened structure 170 may be aligned with the board 110.

As shown in schematic 202, when fabricating the implantable sensor assembly 100, the stiffened structure 170 may be press fit into location through a tolerance fit, to temporarily hold the stiffened structure 170 in place during the manufacturing process. The fastening material 130 may be applied via one or more through apertures (such as through aperture 112), or at least one injection hole (not shown in FIG. 2A) in the stiffened structure 170, thus fastening the stiffened structure 170 to the board 110 with at least one packaged sensors mounted thereon.

At some point in the manufacturing process, such as during a cure time, the viscosity of the fastening material 130 should be such that it can flow into confined spaces, such as the interface between top surface 114 and bottom surface 117, to form a thin layer 172 of the fastening material 130. Thus, it may be desirable for the fastening material 130 to include a first hard epoxy with a first viscosity, and a second hard epoxy with a second viscosity greater than the first viscosity. For example, LOCTITE 3984 is viscous at room temperature. When heated, the viscosity of LOCTITE 3984 decreases allowing for improved flow. Shortly after the viscosity decreases LOCTITE 3984 starts to gain viscosity again and subsequently cures and hardens. Furthermore, it may be desirable for the thin layer 172 of the fastening material 130 to contact one or more metal pads for adhesion 244 on the top surface 114 as the fastening material 130 may have better adhesion with metal surfaces than other materials included in the board 110 (e.g. polyimide). For example, LOCTITE 3984 adheres well to metals.

As shown in schematic 204, the flow of the fastening material 130 may be at least mostly constrained by the geometry of the stiffened structure 170. Specifically, the fastening material 130 should cover at least in part the faces of first packaged sensor 120 other than the central area 126. Thus, when implanted in the body, the first packaged sensor 120 is at least in part exposed to the external environment surrounding the sensor assembly 100 and is thus able to measure a physical condition of the body. It is important to note that portion of the first packaged sensor 120 which is exposed, at least in part, to the external environment by the fastening material 130 may at any point in the manufacturing process be coated in the layer of biocompatible material.

FIG. 2B provides an example of the implantable sensor assembly 100 with improved adhesion and reduced force of insertion. Schematic 210 is a perspective view of the first packaged sensor 120 mounted on the board 110. The one or more metal pads for adhesion 244 are distributed on the top surface 114 of the board 110. Schematic 220 is a perspective view of the stiffened structure 170 (visualized as transparent) joined to the board 110. Schematic 230 and schematic 240 provide examples of possible cross-sectional profiles of the stiffened structure 170 which may reduce insertion force by reducing the cross-sectional area of the forward end 119 as compared to the maximum cross-sectional area of the stiffened structure 170.

As shown in schematic 210 and 220, when fabricating the implantable sensor assembly 100, the stiffened structure 170 may be press fit into location through a tolerance fit, to temporarily hold the stiffened structure 170 in place during the manufacturing process. The set of alignment features 243 and the set of alignment cutouts 242 are configured to align, such that the stiffened structure 170 is fixed in the desired position during the manufacturing process.

### II. Implantable Multi-Sensor Assembly

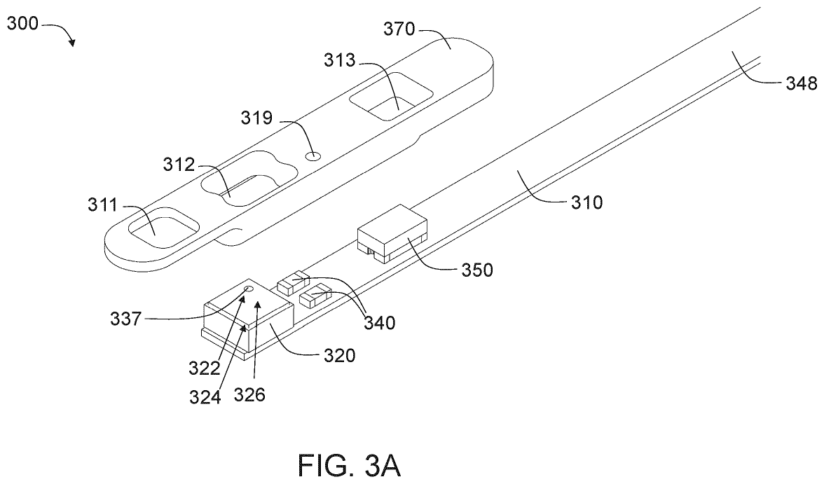

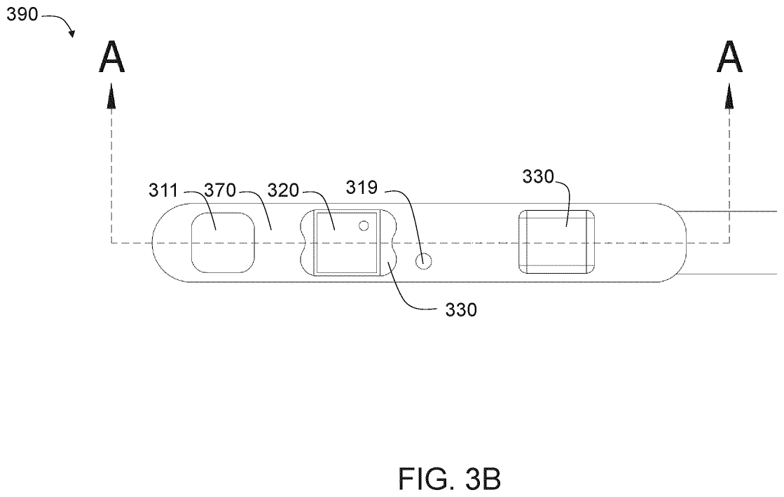

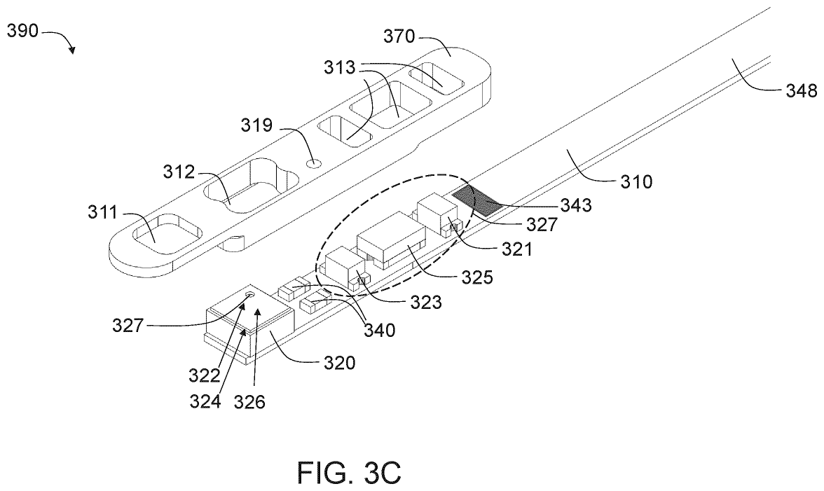

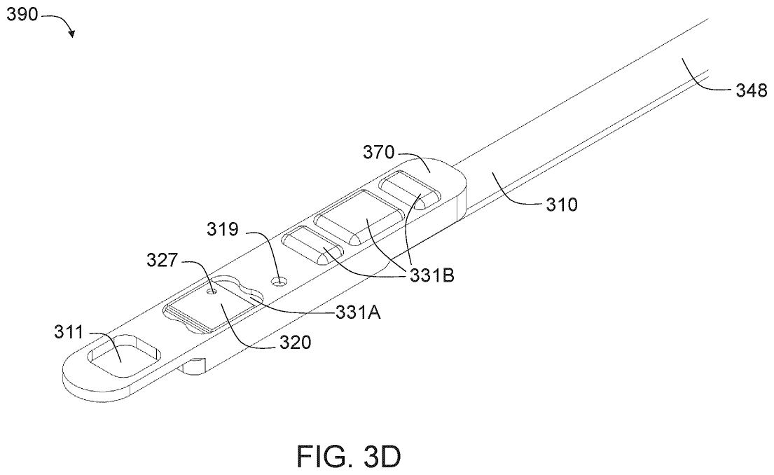

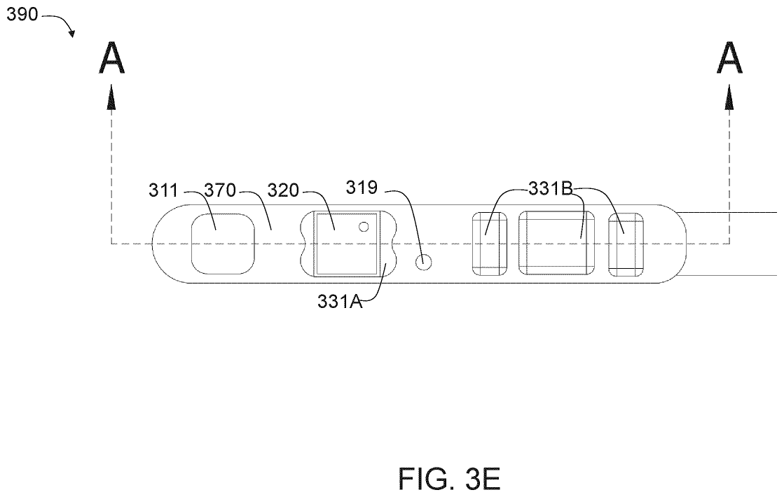

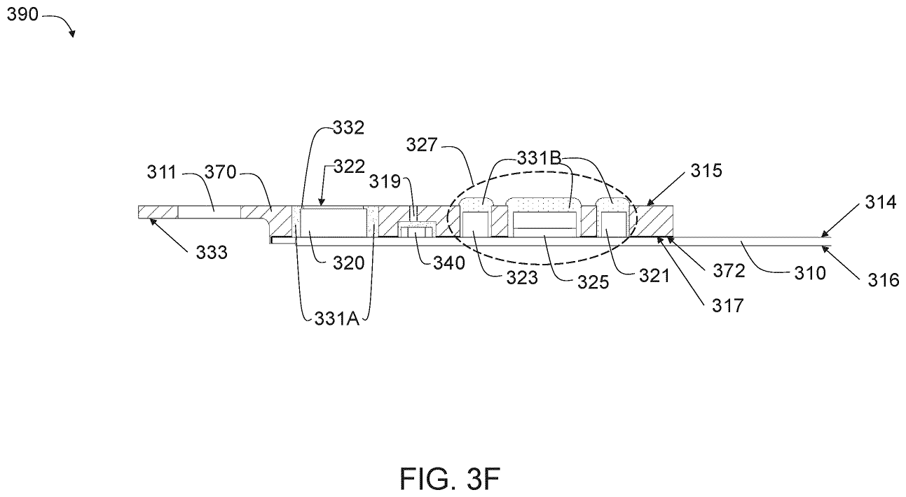

FIG. 3A is an exploded perspective view of an implantable multi-sensor assembly 300. FIG. 3B is a top plan view of an implantable multi-sensor assembly 300.

The implantable multi-sensor assembly 300 includes a board 310, a first packaged sensor 320, a second sensor 350, a stiffened structure 370 and a fastening material 330. The first packaged sensor 320 is mounted to a top surface 314 of the board 310. The first packaged sensor 320 includes a first sensing element 101, an ASIC 102, a supportive medium 103 and an exterior package 104. The first sensing element 101 is operatively connected to the ASIC 102. The second sensor 350 is mounted to the top surface 314 of the board 310. The stiffened structure 370 has a top surface 315 and a bottom surface 317. The stiffened structure 370 has at least one set of through apertures extending between the top surface 315 and the bottom surface 317. A first set of through apertures 312 surrounds the perimeter of the first packaged sensor 320. A second set of through apertures 313 surrounds the perimeter of the second sensor 350. The fastening material 330 joins at least the stiffened structure 370 to the top surface 314 of the board 310. The fastening material 330 material exposes, at least in part, the first packaged sensor 320 and the second sensor 350 to the external environment.

The board 310 provides a surface for mounting at least the first packaged sensor 320 and the second sensor 350. The board 310 is operatively connected to a communication cable 348. The board 310 has the top surface 314 and a bottom surface 316.

The first packaged sensor 320 isolates the sensing element 101 of the first packaged sensor 320 from stress propagating through the implantable multi-sensor assembly 300. The exterior package 104 of the first packaged sensor 320 is rigid to prevent excessive stress on the supportive medium 103. The supportive medium 103 is compliant, and isolates the sensing element 101 and the ASIC 102 from propagating stress. Together the supportive medium 103, the first sensing element 101 and the ASIC 102 may not fill the entire volume inside the exterior package 104, leaving an empty volume 105 in the interior of the first packaged sensor 320. The first packaged sensor 320 has a top face 322 including a central area 326 surrounded by a perimeter 324. Additionally, the first packaged sensor 320 may have at least one hole 337 in the top face 322, to expose the sensing element 101 to external conditions for measurement. To maintain the performance of the first packaged sensor 320, the central area 326 should remain at least mostly free of the fastening material 330. However, lips 332 of the fastening material 330 may extend between the perimeter 324 and the top surface 315.

The stiffened structure 370 is rigid to prevent excessive bending or torsional strain inducing stress in the first packaged sensor 320.

The fastening material 330 is configured to rigidly adhere at least the stiffened structure 370 to the board 310 without failure due to delamination, peeling, shear or tension. Furthermore, the fastening material serves to electrically insulate the electrical components from the external environment.

In some embodiments, the second sensor 350 may include one from a set of (i) an optoelectronic sensor, (ii) a glucose sensor, (iii) a biomarker sensor, (iv) a pH sensor, (v) a temperature sensor. The biomarker sensor may utilize various sensor designs to quantify the concentration of a particular biomarker, now known or later developed. The particular biomarker could include but is not limited to: creatinine kinase, chloride, urea nitrogen, sodium, potassium, bicarbonate, lactic acid and ionized calcium.

FIG. 3C is an exploded perspective view of an implantable multi-sensor assembly 390. FIG. 3D is a perspective view of an implantable multi-sensor assembly 390 of FIG. 3C. FIG. 3E is a top plan view of an implantable multi-sensor assembly 390. FIG. 3F is a cross-sectional, side elevation view of the implantable multi-sensor assembly 390 along the cross-section A-A shown in FIG. 3E.

FIGS. 3C-F depict an example of the implantable multi-sensor assembly 390, where the second sensor 350 is an optoelectronic sensor 327. The optoelectronic sensor 327 measures changes in localized tissue oxygenation within the body by measuring changes in the attenuation of light through tissue. Briefly, the optoelectronic sensor 327 uses in vivo near-infrared spectroscopy (in vivo NIRS) techniques to monitor the concentration of oxygenated hemoglobin (HbO₂) and deoxygenated hemoglobin (Hb). The concentration of HbO₂ and Hb provides information about the localized tissue oxygen saturation levels, rStO₂. Specifically, the localized tissue oxygen saturation level is equivalent to rStO₂≈[HbO₂]/([HbO₂]+[Hb])×100%. One or more sources emit distinct wavelengths of light, while one or more light-sensitive sensors detect the attenuated light backscattered by Hb and HbO₂ in a tissue and produce a voltage signal. Additional details on the operation of the optoelectronic sensor 327 will be provided in the following sections. This approach to measuring highly localized tissue oxygenation may be useful in the monitoring of acute compartment syndrome (ACS) and hypoxia-related conditions as well as monitoring high-risk patients during the perioperative period.

In some embodiments, the optoelectronic sensor 327 further includes: at least one photodiode 325, a first micro-light emitting diode (μLED) 321 and a second μLED 323.

In some embodiments, the at least one photodiode 325, the first μLED 321 and the second μLED 323 include at least one packaged surface mounted technology (SMT) component mounted to the board.

In some embodiments, the at least one photodiode 325 is configured to measure the light emitted at a first wavelength by the first μLED 321 and at a second wavelength by the second μLED 323 and attenuated by a tissue in a body.

In some embodiments, the first wavelength and the second wavelength include one from (i) infrared and (ii) red spectrum. It should be understood that the infrared spectrum encompasses wavelengths from approximately 700 nm to 1 mm, and the red spectrum encompasses wavelengths from approximately 625 nm to 700 nm. In some examples, the first wavelength and the second wavelength are chosen from (i) approximately 660 nm and approximately 940nm, (ii) approximately 625 nm and approximately 540 nm (iii) approximately 645 nm and approximately 950 nm, and (iv) approximately 660 nm and approximately 850 nm.

In some embodiments, the fastening material 330 is configured to scatter light emitted by the first μLED 321 and the second μLED 323.

In some embodiments, the board 310 includes a PCB. The PCB may be rigid or flexible.

In some embodiments, the board 110 further includes one or more metal pads 343 on the top surface of the board 110, and wherein the one or more metal pads 343 are configured to increase the adhesion of the fastening material 330 to the top surface 314 of the board 110, In some embodiments, the top face 322 of the first packaged sensor 320 is at or below the top surface 315 of the stiffened structure 370.

In some embodiments, the first packaged sensor 320 is a surface mounted technology (SMT) sensor and the second sensor 350 is a SMT sensor.

In some embodiments, the first packaged sensor 320 is a pressure sensor, and wherein the sensing element 101 of the first packaged sensor 320 is exposed to the external environment by at least one hole 337 in the top face 322 of the first packaged sensor 320.

In some embodiments, the fastening material 330 is a biocompatible material.

In some embodiments, the fastening material 330 changes viscosity with temperature.

In some embodiments, the fastening material 330 is optically clear and wherein the fastening material 330 is applied, at least in part, to (i) the interface between the stiffened structure 370 and the board 310, and (ii) the interface between the stiffened structure 370 and the exterior package 104 of the first packaged sensor 320, other than the top face 322 of the first packaged sensor 320, such that the top face 322 of the first packaged sensor 320 is at least mostly free of the fastening material 330 and (iii) the interface between the stiffened structure 370 and the second sensor 350, such that the second sensor 350 is completely covered by the fastening material 330.

In some embodiments, the fastening material 330 includes a first hard epoxy 331A and a second hard epoxy 331B, wherein the second hard epoxy 331B is an optically clear epoxy and wherein the first hard epoxy 331A is applied, at least in part, to (i) the interface between the stiffened structure 370 and the board 310, and (ii) the interface between the stiffened structure 370 and the exterior package 104 of the first packaged sensor 320, other than the top face 322 of the first packaged sensor 320, such that the top face 322 of the first packaged sensor 320 is at least mostly free of the first hard epoxy 331A and the second hard epoxy 331B is applied to the interface between the stiffened structure 370 and the second sensor 350, such that the second sensor 350 is completely covered by the second hard epoxy 331B. The first hard epoxy 331A and the second hard epoxy 331B may be formed from one or more materials capable of strong bonding to metallic surfaces, such as LOCTITE-3984 from Henkel, MED-302-3M from Epo-Tek, EP30-4Med from Masterbond, or EP42HT-4AOMed from Masterbond. Additionally, due to the envisioned medical application of the implantable multi-sensor assembly 300, the first hard epoxy 331A and the second hard epoxy 331331B may be biocompatible.

In some further embodiments, the first hard epoxy 331A has a first viscosity, the second hard epoxy 331B has a second viscosity, and either (i) the first viscosity is greater than the second viscosity or (ii) the second viscosity is greater than the first viscosity.

In some embodiments, the stiffened structure 370 is configured to enable the fastening material 330 to be applied via one or a combination of (i) one or more injection holes 319 and (ii) at least one set of through apertures, and wherein the flow of the fastening material 330 is constrained, at least in part, by the stiffened structure 370 and the board 310.

In some embodiments, a forward end 333 of the stiffened structure 370 further includes a hook hole 311, wherein the hook hole 311 is adapted for interfacing with an insertion device.

In some embodiments, the cross-sectional area of the forward end 333 of the stiffened structure 370 is less than the maximum cross-sectional area of the stiffened structure 370.

In some embodiments, the stiffened structure 370 is made from a biocompatible material. For example, the stiffened structure 360 may be stainless steel or titanium.

In some embodiments, the implantable sensor assembly 300 is conformally coated in a layer of biocompatible material, wherein the layer of biocompatible material conformally coats, at least partly, the interior of the first packaged sensor, and wherein the layer of biocompatible material is one from a selection of (i) parylene, (ii) a biocompatible epoxy and (iii) a biocompatible gel.

In some embodiments, one or both of the first packaged sensor 320 and the second sensor 350 are conformally coated in a layer of biocompatible material, wherein the layer of biocompatible material conformally coats, at least partly, the interior of the first packaged sensor, and wherein the layer of biocompatible material is one from a selection of (i) parylene, (ii) a biocompatible epoxy and (iii) a biocompatible gel.

In some embodiments, the implantable multi-sensor assembly 300 further includes: at least one peripheral SMT component 340 mounted on the board 310 and operatively connected to the first packaged sensor 320; and a communication cable 348 extending from the board 310 and adapted to operatively connect at least the first packaged sensor 320 and the second sensor 350 to a data collection module (not shown).

In some further embodiments, the board 310 and the communication cable 348 are the same PCB, wherein the PCB is flexible. For example, the PCB may be mostly made of polyimide.

In some embodiments, the electrical connections between one or a combination of (i) the at least one peripheral SMT component 340, (ii) the second sensor 350 and (iii) the first packaged sensor 320 include one or more elements selected from wire bonds, solder paste traces on the board, conductive ink traces on the board, conductive metal traces on the board, conductive epoxy traces on the board, and a combination thereof.

In some embodiments, the second sensor 350 is made up of a plurality of components mounted to the board 310, and wherein each respective through aperture from the second set of through apertures surrounds the perimeter of a respective component from the plurality of components mounted to the board 310.

In some examples, the stiffened structure 370 further includes at least one injection hole 319, wherein each of the at least one injection hole 319 provides a through aperture from the top face 315 of the stiffened structure 370 to a cavity on the bottom face 117 of the stiffened structure 370.

FIG. 3F provides an example of how the implantable multi-sensor assembly 390 is configured to control the path of light emitted from the first μLED 321 and the second μLED 323. The height of the stiffened structure is such that light emitted by the first μLED 321 and the second μLED 323 cannot reach the at least one photodiode 325 via a linear path. In other words, light emitted by the first μLED 321 and the second μLED 323 must be scattered by a tissue to reach the at least one photodiode 325. Additionally, the material properties and cure conditions of the fastening material 330 may be chosen such that the curvature of the interface of the fastening material 330 is increased, increasing the scattering of light into the tissue.

It will be understood that in some scenarios, measuring only the localized tissue oxygen saturation level with the optoelectronic sensor 327 may be desirable. In these cases, it is straightforward to eliminate the first packaged sensor 320, the electrical connections of the first packaged sensor 320 and the first set of through apertures 312 from the implantable multi-sensor assembly 390.

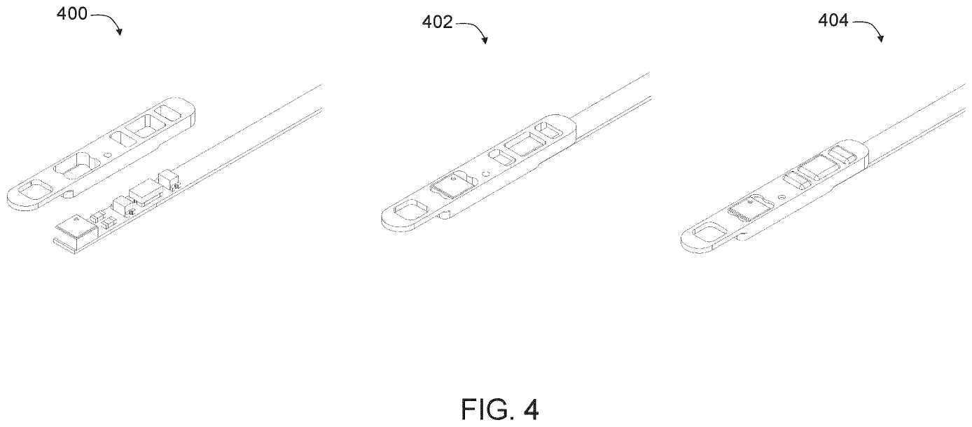

FIG. 4 provides an overview of the assembly steps of the implantable sensor assembly 390. Schematic 400 is a perspective view of an implantable multi-sensor assembly 390 before assembly. Schematic 402 is a perspective view of an implantable multi-sensor assembly 390 before fastening material is applied. Schematic 404 is a perspective view of an implantable multi-sensor assembly 390 after fastening material is applied.

As shown in schematic 400, when fabricating the implantable multi-sensor assembly 390, the stiffened structure 370 may be aligned with the board 310. As shown in schematic 402, when fabricating the implantable multi-sensor assembly 350, the stiffened structure 370 may be press fit into location through a tolerance fit, to temporarily hold the stiffened structure 370 in place during the manufacturing process. The fastening material 330 may be introduced via one or more through apertures (such as through aperture 312), or at least one injection hole 319 in the stiffened structure 370, thus fastening the stiffened structure 370 to the board 310 with at least one packaged sensors mounted thereon.

At some point in the manufacturing process, such as during a cure time, the viscosity of the fastening material 330 should be such that it can flow into confined spaces, such as the interface between top surface 314 and bottom surface 317, to form a thin layer 372 of the fastening material 330. Thus it may be desirable for the fastening material 330 to include a first hard epoxy 331A with a first viscosity, and a second hard epoxy 331B with a second viscosity, wherein either (i) the first viscosity is greater than the second viscosity or (ii) the second viscosity is greater than the first viscosity.

As shown in schematic 404, the flow of the fastening material 330 may be at least mostly constrained by the geometry of the stiffened structure 370. Specifically, the fastening material 330 should cover at least in part the faces of first packaged sensor 320 other than the central area 326 that is exposed to the through aperture 312. Thus, when implanted in the body, the first packaged sensor 320 is at least in part exposed to the external environment surrounding the multi-sensor assembly 390 and is thus able to measure a physical condition of the body. It is important to note that portion of the first packaged sensor 320 which is ‘in part exposed to the external environment’ by the absence of fastening material 330 may at any point in the manufacturing process be coated in the layer of biocompatible material.

### III. Optoelectronic Sensor Signal Generation and Processing

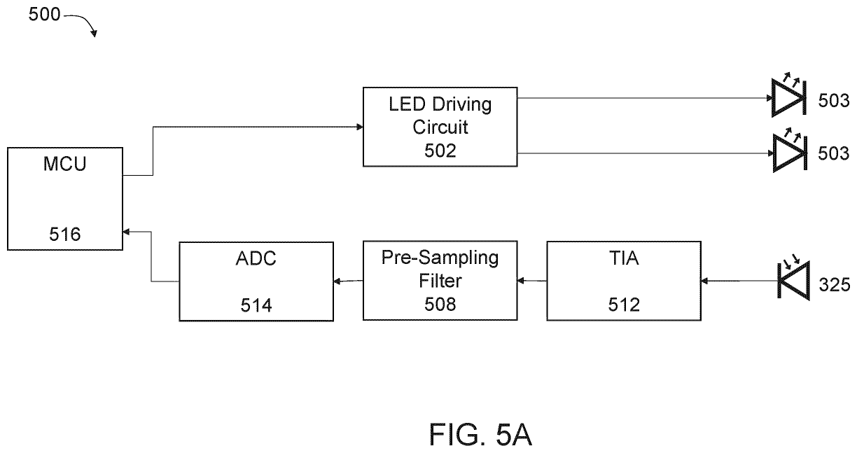

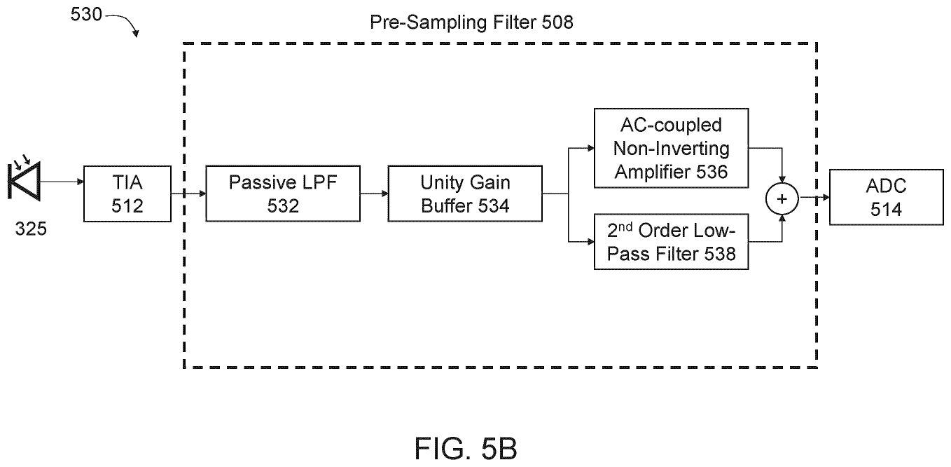

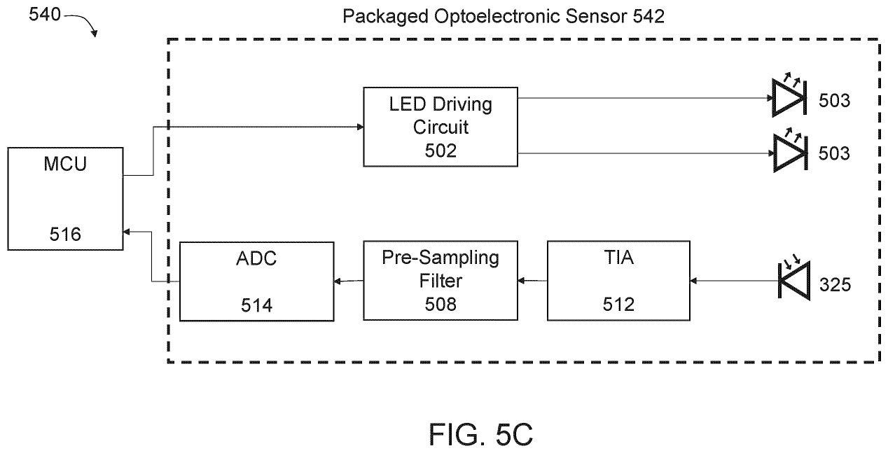

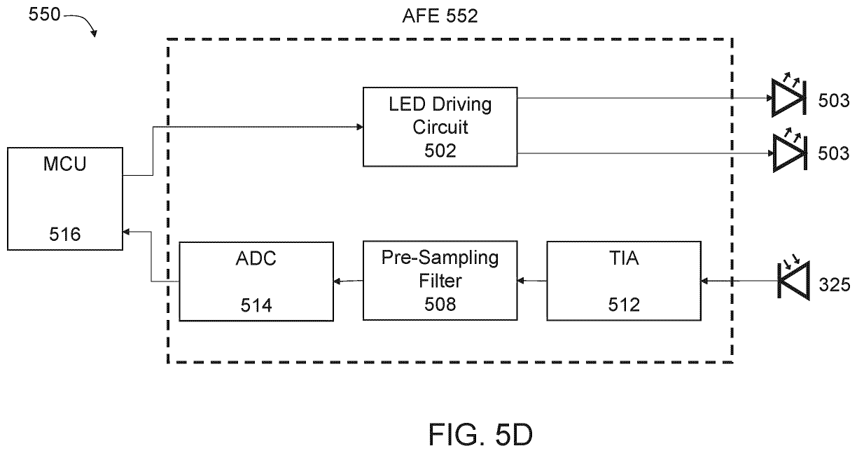

FIG. 5A provides an example of a circuit 500 for signal generation and processing, for use with an optoelectronic sensor 327. The circuit 500 includes a micro-processor unit (MCU) 516 which is operatively connected to an LED driving circuit 502 and further operatively connected to two or more LEDs 503. In some examples, the two or more LEDs 503 may include the first μLED 321 and the second μLED 323. The circuit 500 also includes at least one photodiode 325 which is operatively connected to a TIA 512, which is further operatively connected to a pre-sampling filter circuit 508, which is further operatively connected to an analog-to-digital converter (ADC) 514, which is further operatively connected to the MCU 516. In some examples, the MCU 516 may further include the ADC 514. In some examples, the MCU 516 and ADC 514 may be separate components.

The MCU 516 controls the operation of the one or more LEDs 503 with the LED driving circuit 502. The two or more LEDs 503 emit light at a first wavelength and a second wavelength. The at least one photodiode 325 generates a raw current signal based on the quantity of light absorbed at the first wavelength and at the second wavelength. The TIA 512 converts the raw current signal to a voltage signal. The pre-sampling filter circuit 508 removes low and high frequency noise. The ADC 514 converts the analog voltage signal to a digital signal. The MCU 516 further cleans, filters, calibrates, processes and transmits the signal.

FIG. 5B provides an example of a circuit 530 for signal processing, for use with the signal generated by the at least one photodiode 325. The circuit 530 includes a TIA 512 which is operatively connected to the pre-sampling filter 508, which is operatively connected to the ADC 514. The pre-sampling filter 508 includes a passive low-pass filter (LPF) 532 which is operatively connected to a unity gain buffer 534 which is operatively connected to both an AC-coupled non-inverting amplifier 536 and a second-order low-pass sallen-key filter 538. In the circuit 530, each of the at least one photodiode 325 is operated without bias applied (photovoltaic mode) and outputs a current signal. The TIA 512 converts the current signal to a voltage signal. The passive LPF 532 removes high-frequency noise above a target frequency. The unity gain buffer 534 isolates the components of the signal before and after the unity gain buffer 534. The signal output from the unity gain buffer 534 is fed into an AC-coupled non-inverting amplifier 536 with a high gain to isolate and amplify the AC components of the signal. The signal output from the unity gain buffer 534 is also fed into the second-order low-pass sallen-key filter 538 with a very low cutoff to isolate only the DC offset of the signal. The output of the AC-coupled non-inverting amplifier 536 and the second-order low-pass sallen-key filter 538 are added together to obtain the filtered DC component with the amplified AC components which are below the particular frequency. The resultant signal is input to the ADC 514.

In some examples, the passive LPF 532 removes high-frequency noise above the particular frequency >5 Hz. In some further examples, the passive LPF 532 removes high-frequency noise above the particular frequency >2 Hz. In some further examples, the passive LPF 534 removes high-frequency noise above the particular frequency >1 Hz. In yet further examples, the passive LPF 534 removes the noise beyond approximately 0.2-2 Hz, which is the noise associated with the heart rate and respiration. Thus the signal indicative of heartrate can be isolated and amplified if desired.

In some examples, the second-order low-pass sallen-key filter 538 has a cutoff of 0.5 Hz. In some examples, the second-order low-pass sallen-key filter 538 has a cutoff of 0.2 Hz. In some further examples, the second-order low-pass sallen-key filter 538 has a cutoff of 0.02 Hz. In yet further examples, the second-order low-pass sallen-key filter 538 has a cutoff such that the DC component associated with an average tissue oxygenation is separable from the AC component associated with heartrate.

FIG. 5C provides an example of a circuit 540 for signal generation and processing applied in combination with a packaged optoelectronic sensor 542. The example circuit 540 includes the MCU 516 which is operatively connected to the packaged optoelectronic sensor 542. The packaged optoelectronic sensor 542 includes the LED driving circuit 502 and the at least one LEDs 503 required to emit light at the first wavelength and the second wavelength. The packaged optoelectronic sensor 542 also includes the at least one photodiode 325 which is operatively connected to a TIA 512, then further operatively connected to a pre-sampling filter circuit 508, then further operatively connected to the ADC 514. The amount and type of filtering, processing and signal conditioning will depend on the packaged optoelectronic sensor 542. In some examples, the packaged optoelectronic sensor 542 may include an optical biosensor like BH1790GLC from ROHM Semiconductor, or the OB1203 from Renesas. In some examples, the packaged optoelectronic sensor 542 may comprise a SMT device.

FIG. 5D provides an example of a circuit 550 for signal generation and processing in combination with a pre-packaged analog front-end (AFE) 552. The example circuit 550 includes the MCU 516 which is operatively connected to the AFE 552, which is further operatively connected to the at least two LEDs 503 and the at least one photodiode 325. The AFE 552 includes the LED driving circuit 502. The AFE 552 also includes the TIA 512, which is operatively connected to a pre-sampling filter circuit 508, then further operatively connected to the ADC 514.

In some alternative examples, the AFE 552 and the LED driving circuit 502 may be separate. In some alternative examples, the AFE 552 and the ADC 514 may be separate.

In some examples, the LED driving circuit 502 may offer controllable LED intensity. For example, analog or pulse-width modulation (PWM) dimming may be possible by attaching the LED driving circuit 502 to an appropriate pin of the MCU 516 and changing the current driven through the first μLED 321 and second μLED 323. In the case of analog dimming, a DAC 518 varies the DC current supplied to the LED driving circuit 502. In this case, a DAC analog output pin would be connected to the LED driving circuit 502 directly. In the case of PWM dimming the MCU 516 varies the duty cycle of a constant DC current, thus altering the average DC current. In this case, the appropriate pin of the MCU 516 may switch on/off a current-providing circuit.

In some examples, controllable LED intensity may be used to improve signal processing. For example, by monitoring the signal sampled from the ADC 514, the LED intensity can be altered to increase/decrease the voltage signal generated by the photodiode circuit 504. Altering the intensity of the at least two LEDs 503 can prevent signal saturation of the TIA 512 amplifier or the ADC 514. Thus, it is possible to control the magnitude of the at least one photodiode 325 current, by controlling the LED brightness via the MCU 516. In the envisioned application (in vivo NIRS) control over signal saturation is critical as the sensor is inserted into the body which is not a controlled environment. Variables such as tissue type, region in the body, fat content and other differences between individuals, will affect the baseline absorption of the emitted light, thus control over the brightness of the at least two LEDs 503 ensure that information is not lost due to signal saturation.

Additionally, the control scheme of the LED driving circuit 502 may be varied. In some examples, the LED driving circuit 502 of the first μLED 321 may be connected to a first general-purpose input/output (GPIO) pin 526 of the MCU 516, and the second μLED 323 may be connected to a second GPIO pin 528 of the MCU 516. The pin state of the first GPIO pin 526 and the second GPIO pin 528 may be controlled by MCU 516, such that the desired μLEDs are illuminated. In some examples, the LED driving circuit 502 of the first μLED 321 and the second μLED 323 may be connected to a single GPIO pin 530. In this example, the first μLED 321 and the second μLED 323 may be controlled by a simple oscillating signal from the MCU 516. Various hardware implementations may be used to transmit the oscillating signal, including bit-banging and pulse-width modulation. In some examples, the LED driving circuit 502 may include timer based control signals. In some examples, the LED driving circuit 502 may incorporate delayed control signals, to allow for component set-up and settling, or to incorporate ambient light measurements, or to establish a baseline voltage signal (i.e. dark current) generated by the photodiode circuit 504 prior to illumination of the first μLED 321 and the second μLED 323.

In some examples, the photodiode circuit 504 may be wired in reverse or forward biased configurations. In the reverse bias configuration (photoconductive mode), the voltage offset can be applied to the photodiode circuit 504. The reverse bias configuration gives a faster response but leads to a dark current.

In some examples, the photodiode circuit 504 may have no bias applied (photovoltaic mode). Photovoltaic mode may give slower response time to changes in light level but gives very small dark current which leads to better noise characteristics.

In some examples, the transimpedance amplifier (TIA) 512, may have either a fixed or a variable gain and alternative upstream or downstream circuitry. In one example, a current amplifier 520 may be added immediately following the photodiode circuit 504 but before the TIA 512. In one example, a low noise amplifier (LNA) 522 may be added immediately following the photodiode circuit 504. In other examples, combinations of these upstream and downstream circuitry may be combined. The choice of upstream and downstream circuitry is dependent on factors including; the type of μLEDs (wavelength, light intensity etc.), the sensitivity of the photodiode, the arrangement of the μLEDs with respect to the at least one photodiode 325, the anticipated location of the sensor with respect to the body as well as many others.

The pre-sampling filter circuit 508 isolates specific frequencies around one or more target frequencies. The pre-sampling filter 508 may be configured to exclude high frequency noise from power line frequency and associated harmonics. In some examples, the pre-sampling filter 508 isolates the ultra-low (DC) frequency to measure an average localized tissue oxygenation level (the DC component providing a time average of the localized tissue oxygenation level). As a consequence, the pre-sampling filter 508 may also be configured to exclude the frequencies associated with the respiratory and heart rates (~0.2-2 Hz) Alternately, if it is desirable to measure the respiratory or heart rates, the pre-sampling filter 508 may be configured to isolate only frequencies in the ~0.2-2 Hz range. Furthermore, the one or more target frequencies may be separated, filtered or processed separately, and recombined to obtain an overall signal that is indicative of both the localized tissue oxygenation level and the heart rate/respiration rate. Additionally, the pre-sampling filter 508 may be configured to isolate a frequency associated with the pulse frequency of the first μLED 321 and/or the second μLED 323. In some examples, bandpass filtering may be used to obtain an approximate bandwidth. Bandpass filtering may be implemented with cascaded low-pass filters and high-pass filters, or implemented with a designated bandpass filter. In some examples, the pre-sampling filter circuit 508 may be used in addition to digital filtering implemented by the MCU 516. In some examples, digital filtering implemented by the MCU 516 may be configured to remove low frequency noise from sources like ambient light.

In some examples, a second stage amplifier 524 may be implemented immediately before the MCU 516 to make use of the full-scale voltage of the ADC 514. By utilizing at least most of the full-scale voltage swing of the ADC 514, the impact of the ADC 514 quantization error may be limited. The ADC 514 quantization error is a byproduct of the full-scale voltage and resolution of the ADC 514. In some embodiments, the second stage amplifier 524 may be an AC-coupled non-inverting amplifier, whose desired gain and cutoff frequency is dependent on many factors, including; the frequency of the voltage signal, the amplitude of the voltage signal, the signal-to-noise (SNR) of the voltage signal and the reference voltage and resolution of the ADC 514.

In some examples, the TIA 512 may be a variable gain TIA, wherein the gain of the amplifier is dependent on the signal characteristics sampled by the ADC 514. In this example, there may be multiple TIA feedback resistors, whose connection to the TIA 512 is controlled using a switch, for example a single-pole single-throw (SPDT) switch or a multiplexer. Altering the gain of the amplifier can prevent signal saturation of the TIA 512 amplifier or the ADC 514, thus improving sampled signal quality.

### IV. Modular Implantable Sensor Assemblies

The implantable sensor assemblies and methods for manufacturing them, as described herein, may be further utilized to create modular implantable sensor assemblies. Herein and throughout this disclosure ‘modular’ means that the implantable sensor assembly design includes a packaged sensor which is surface mounted to the board, and which may be exchanged with another packaged sensor, while retaining substantially the same board, stiffened structure and method of manufacturing.

A modular implantable sensor assembly includes a board, a set of at least one packaged sensor, a stiffened structure and a fastening material. Each respective packaged sensor from the set of at least one packaged sensor is mounted to a top surface of the board. The stiffened structure further includes a set of at least one through apertures between a top surface of the stiffened structure and a bottom surface of the stiffened structure. Each respective through aperture from the set of at least one through apertures surrounds the perimeter of a respective packaged sensor from the set of at least one packaged sensor. The fastening material joins the stiffened structure to the top surface of the board. The fastening material exposes, at least in part, each respective packaged sensor from the set of at least one packaged sensor to the external environment.

The set of at least one packaged sensor includes one or more from a set of (i) an optoelectronic sensor, (ii) a glucose sensor, (iii) a biomarker sensor, (iv) a pH sensor, (v) a temperature sensor, and (vi) a pressure sensor, etc.

Therefore, it will be understood that a modular implantable sensor assembly may include one or more different packaged sensors of the same or of different types, where the stiffened structure includes respective through aperture(s) to accommodate the sensors, without departing from the present teachings.

Herein and throughout this disclosure, ‘exposes, at least in part’ means that the fastening material at least exposes each respective packaged sensor to the phenomenon being sensed by the respective packaged sensor. For example, the fastening material may fully cover a photodiode, so long as the fastening material is optically clear and light may pass through it for sensing. In another example, a rigid and impermeable fastening material may only cover the sides of a pressure sensor, such the top face with a hole connected to the interior of the pressure sensor, remains uncovered for sensing. Furthermore, an impermeable fastening material may not fully cover a field-effect transistor-based biomarker sensor.

### V. Insertion

In order to provide useful measurements of conditions within a body, it is necessary to implant a sensor assembly within a region of interest. The implantable sensor assemblies (100, 300, 390) described herein are possible to implant in the body without the need for first providing a surgical incision, although all of the implantable sensor assemblies may also be inserted through a surgical incision if desired. Furthermore, the skilled reader will appreciate that the following information may be applied to all the implantable sensor assemblies (100, 300, 390) described herein, though implantable sensor assembly 100 is used as an example.

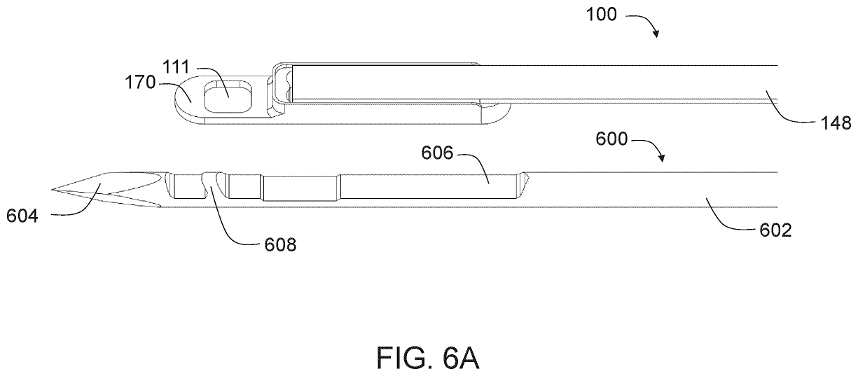

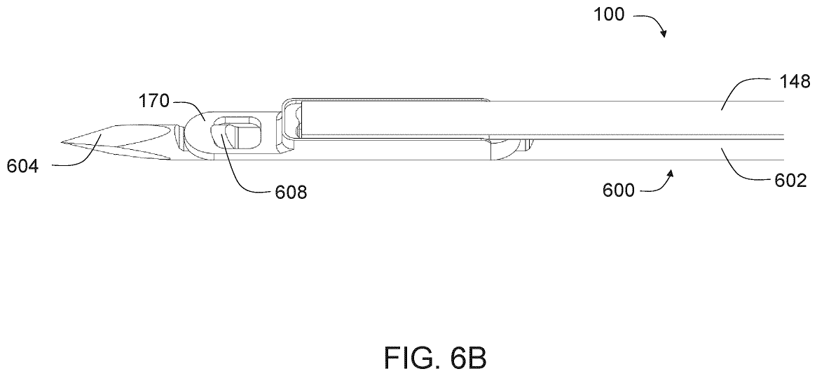

FIG. 6A is a perspective view of the implantable sensor assembly 100 and of a forward end of an insertion device 600, unassembled. The forward end of the insertion device 600 includes an elongated member 602. The elongated member 602 terminates at a puncturing end 604 for puncturing skin to allow insertion of the implantable sensor assembly 100 in a body. The stiffened structure 170 includes the hook hole 111 which allows the implantable sensor assembly 100 to connect to the insertion device 600.

FIG. 6B is a perspective view of the implantable sensor assembly 100 and of the forward end of the insertion device 600, assembled. A forward-facing hook 608 protrudes from the recess 606 and is sized, positioned and configured for being inserted in the hook hole 111 of the stiffened structure 170 when the sensor assembly 100 is received in the recess 606 of the insertion device 600. The mating of the hook 608 and of the hook hole 111 provides for maintaining a connection of the implantable sensor assembly 100 and of the insertion device 600 when assembled. After insertion of the implantable sensor assembly 100 and of the forward end of the insertion device 600 in the body, the implantable sensor assembly 100 may be disengaged from the insertion device 600 via an approximately 180 degree rotation followed by retraction of the insertion device 600. After implantation, the implantable sensor assembly 100 may be removed from the body when desired by gently pulling on the communication cable 148.

The board 110 is further operatively connected to the communication cable 148. The board translates signals generated by the first packaged sensor 120 and peripheral SMT components 140 to a data collection module (not shown). The data collection module is located outside the body. The data collection module may receive signals via the communication cable 148, receive signals from other sources, store signal data, condition, process and combine signals and send signals to other sources.

The implantable sensor assembly 100 and the implantable multi-sensor assembly 300/390 are sized for insertion under the skin of a patient, without requiring a previous incision or blunt dissection. In general, an overall width ‘W’ of the sensor assembly 100 does not exceed about 2.4 mm, an overall height ‘H’ of the sensor assembly 100 does not exceed about 1.35 mm, and an overall length ‘L’ of the sensor assembly as measured from a rear-end of stiffened structure 170/370 to a forward-end of the stiffened structure 170/370 does not exceed about 11.49 mm.

The elongated member 602 is sized for insertion to at least the deep posterior compartment of the lower limb, such that the implantable sensor assembly 100 may be implanted in the deep posterior compartment.

Those of ordinary skill in the art will realize that the description of the implantable sensor assembly is illustrative only and are not intended to be in any way limiting. Other embodiments will readily suggest themselves to such persons with ordinary skill in the art having the benefit of the present disclosure. Furthermore, the disclosed implantable sensor assembly may be customized to offer valuable solutions to existing needs and problems related to the exposure of implantable biocompatible sensors to various physical conditions. In the interest of clarity, not all of the routine features of the implementations of the implantable sensor assembly are shown and described. In particular, combinations of features are not limited to those presented in the foregoing description as combinations of elements listed in the appended claims form an integral part of the present disclosure. It will, of course, be appreciated that in the development of any such actual implementation of the implantable sensor assembly, numerous implementation-specific decisions may need to be made in order to achieve the developer's specific goals, such as compliance with application-related, system-related, and business-related constraints, and that these specific goals will vary from one implementation to another and from one developer to another. Moreover, it will be appreciated that a development effort might be complex and time-consuming, but would nevertheless be a routine undertaking of engineering for those of ordinary skill in the field of implantable sensors having the benefit of the present disclosure.

The present disclosure has been described in the foregoing specification by means of non-restrictive illustrative embodiments provided as examples. These illustrative embodiments may be modified at will. The scope of the claims should not be limited by the embodiments set forth in the examples, but should be given the broadest interpretation consistent with the description as a whole.

1. An implantable sensor assembly for taking measurements within the body, comprising:
a board;
a first packaged sensor, mounted to a top surface of the board, the first packaged sensor comprising:
a first sensing element;
an integrated circuit component, wherein the first sensing element is operatively connected to the integrated circuit component;
a supportive medium; and
an exterior package containing the first sensing element, the integrated circuit component and the supportive medium;
a stiffened structure, wherein the stiffened structure further comprises at least one set of through apertures between a top surface of the stiffened structure and a bottom surface of the stiffened structure, wherein a first set of through apertures of the stiffened structure surrounds the perimeter of the first packaged sensor; and
a fastening material, wherein the fastening material joins at least the stiffened structure to the top surface of the board and wherein the fastening material exposes, at least in part, the first packaged sensor to an external environment.

2. The sensor assembly of claim 1, wherein the integrated circuit component is an application-specific integrated circuit (ASIC).

3. The sensor assembly of claim 1, wherein the board comprises a printed circuit board (PCB).

4. The sensor assembly of claim 1, wherein the board further comprises one or more metal pads on the top surface of the board, and wherein the one or more metal pads are configured to increase the adhesion of the fastening material to the top surface of the board.

5. The sensor assembly of claim 1, wherein a top face of the first packaged sensor is at or below the top surface of the stiffened structure.

6. The sensor assembly of claim 1, wherein the first packaged sensor comprises a surface mounted technology (SMT) sensor.

7. The sensor assembly of claim 1, wherein the first packaged sensor comprises a pressure sensor, and wherein the first sensing element of the first packaged sensor is exposed to the external environment by at least one hole in the top face of the first packaged sensor.

8. The sensor assembly of claim 1, wherein the fastening material is applied, at least in part, to (i) the interface between the stiffened structure and the board, and (ii) the interface between the stiffened structure and the exterior package of the first packaged sensor, other than the top face of the first packaged sensor, such that the top face of the first packaged sensor is at least mostly free of the fastening material.

9. The sensor assembly of claim 1, wherein the fastening material comprises a biocompatible material.

10. The sensor assembly of claim 1, wherein the fastening material comprises at least one hard epoxy.

11. The sensor assembly of claim 10, wherein the at least one hard epoxy further comprises a first hard epoxy with a first viscosity, and a second hard epoxy with a second viscosity greater than the first viscosity.

12. The sensor assembly of claim 1, wherein the stiffened structure is configured to enable the fastening material to be applied via one or a combination of (i) one or more injection holes and (ii) at least one set of through apertures, and wherein the flow of the fastening material is constrained, at least in part, by the stiffened structure and the board.

13. The sensor assembly of claim 1, wherein a forward end of the stiffened structure further comprises a hook hole, and wherein the hook hole is adapted for interfacing with an insertion device.

14. The sensor assembly of claim 1, wherein the cross-sectional area of the forward end of the stiffened structure is less than the maximum cross-sectional area of the stiffened structure.

15. (canceled)

16. The sensor assembly of any one of claim 1, wherein the sensor assembly is conformally coated in a layer of a biocompatible material, wherein the layer of biocompatible material conformally coats, at least partly, the interior of the first packaged sensor, and wherein the layer of biocompatible material is one from a selection of (i) parylene, (ii) a biocompatible epoxy and (iii) a biocompatible gel.

17. The sensor assembly of any one of claim 1, wherein the first packaged sensor is conformally coated in a layer of biocompatible material, wherein the layer of biocompatible material conformally coats, at least partly, the interior of the first packaged sensor, and wherein the layer of biocompatible material is one from a selection of (i) parylene, (ii) a biocompatible epoxy and (iii) a biocompatible gel.

18. The sensor assembly of claim 1, further comprising:
at least one peripheral SMT component, mounted to the board, wherein the at least one peripheral SMT component is operatively connected to the first packaged sensor; and
a communication cable extending from the board and adapted to operatively connect the first packaged sensor to a data collection module.

19. The sensor assembly of claim 18, wherein the board and the communication cable comprise the same PCB, wherein the PCB is flexible.

20. The sensor assembly of claim 18, wherein electrical connections between the at least one peripheral SMT component and the first packaged sensor each comprise one or more elements selected from wire bonds, solder paste traces on the board, conductive ink traces on the board, conductive metal traces on the board and conductive epoxy traces on the board.

21. A method of manufacturing the sensor assembly, comprising:
providing a board with at least one packaged sensors mounted thereon;
aligning a stiffened structure with the at least one packaged sensors such that the at least one packaged sensors fit into one or more through apertures of the stiffened structure; and
fastening the stiffened structure to the board with at least one packaged sensors mounted thereon.
